(19) United States
(12) Plant Patent Application Publication
(10) Pub. No.: US 2023/0329130 P1
(43) Pub. Date: Oct. 12, 2023

(54) Cannabis Plant Named 'DRG2'

(50) ;

(71) Applicant: Michael Ray Fowler, McKinleyville, CA (US)

(72) Inventor: Michael Ray Fowler, McKinleyville, CA (US)

(21) Appl. No.: 18/445,192

(22) Filed: May 18, 2023

Related U.S. Application Data
(63) Continuation-in-part of application No. 17/369,828, filed on Jul. 7, 2021.
(60) Provisional application No. 63/049,566, filed on Jul. 8, 2020.

(51) Int. Cl. A01H 6/28

(52) U.S. Cl. PLT/258

(57) ABSTRACT

The unique annual herbaceous Cannabis plant variety ‘DRG2’ is provided. ‘DRG2’ is intended for use as a medicinal herb for sale in Cannabis dispensaries, and was developed for PTSD, stress, and anxiety.

### CROSS REFERENCE TO RELATED APPLICATIONS

The present Application for Patent is a continuation in-part of U.S. application Ser. No. 17/369,828 filed on Jul. 7, 2021, which in turn claims the benefit of Provisional Application No. 63/049,566 entitled “CANNABIS PLANT NAMED ‘DRG2’” filed Jul. 8, 2020, and hereby expressly incorporated by reference herein.

Latin name of the genus and species: Genus—Cannabis. Species—sativa.

### VARIETY DENOMINATION

The new Cannabis plant claimed is of the variety denominated ‘DRG2’.

### BACKGROUND OF THE INVENTION

### Field of the Invention

The present invention relates to a new and distinct annual variety of C. sativa, which has been given the variety denomination of ‘DRG2’. ‘DRG2’ is intended for use as a medicinal herb for sale in Cannabis dispensaries.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense, legal commercialization in the United States as industrial hemp for a variety of purposes including biodegradable plastics and building materials, clothing, paper, food, fuel, and medicines.

Cannabidiol (CBD) extracted from Cannabis widely used in over-the-counter medicines and topical treatments, and is also the active ingredient in the FDA-approved drug Epidiolex. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of C. sativa, which has been given the variety denomination of ‘DRG2’. ‘DRG2’ is intended for use as use as a medicinal herb for sale in Cannabis dispensaries, developed for post-traumatic stress disorder (PTSD), stress, and anxiety.

The new C. sativa variety is a selection resulting from pollen breeding of a '06 Santa Cruz ‘Blue Dream’ female (not patented) with ‘Old Dave’ (a Pre '88 DRG Skunk OG heirloom male; not patented). The new variety was discovered and selected as a single plant within a population of 200+ C. sativa hybrid plants resulting from this controlled pollination in 2007 in an outdoor, full-sun garden at Mendocino County, CA. Selection criteria included isolation of a single female with superior traits from both parents and an uplifting effect that reduces pain.

Asexual reproduction of the new variety by cutting propagation since 2007 in Mendocino County, CA has demonstrated that the new variety reproduces true to type with all of the characteristics, as herein described, firmly fixed and retained through successive generations of such asexual propagation. A stable seed line of the new variety has also been developed by successive generations of interbreeding and selection to correspond to the phenotypic characteristics described herein.

The following characteristics of the new variety have been repeatedly observed and can be used to distinguish ‘DRG2’ as a now and distinct variety of C. sativa hybrid plant:
1. Distinct “Blue” flower sheen with a blue/green leaf color during growth.
2. An obvious “Skunk” nose in both live and dry flowers, as well as the aroma on combustion.
3. Flower has a full color pallet range when cultivated properly.
4. Elongated “sativa” flower buds that have the density of an heirloom Skunk OG.

Plants of the new variety differ from plants of the seed parent ‘Blue Dream’ primarily in plant structure, bud density, and flower aroma. Plants of the new variety differ from plants of the pollen parent ‘Old Dave’ in plant structure, bud size, and flower color. The ‘DRG2’ variety takes the best attributes of both parents, combining a cerebral uplifting high of the mother with a defined pain relief effect of the father.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying photographic illustrations show the typical appearance of the new variety ‘DRG2’. The colors are as nearly true as is reasonably possible in a color representation of this type. Colors in the photographs may differ slightly from the color values cited in the detailed botanical description, which accurately describes the colors of the new plant.

FIG. 1 is a photograph of the new variety ‘DRG2’ at 60 days indoor aeroponic.

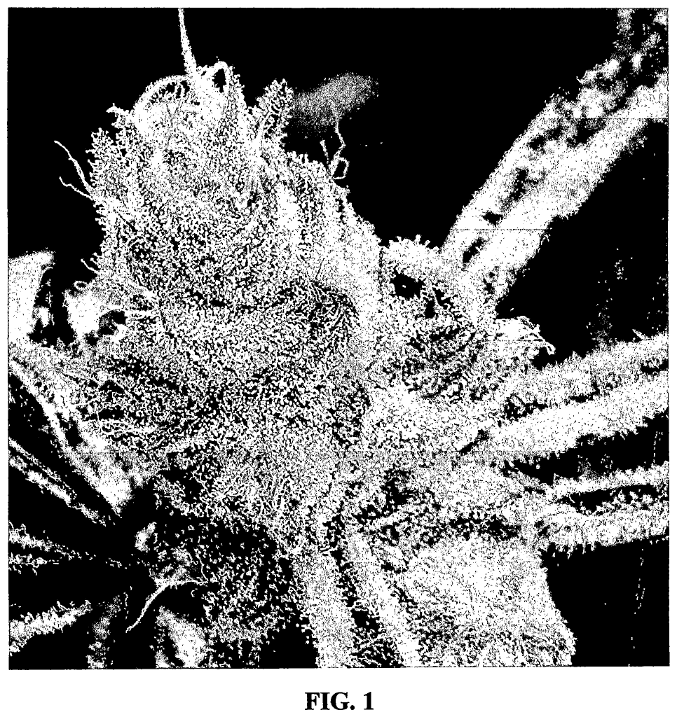

FIG. 2 is a photograph of the new variety ‘DRG2’ at 60 days indoor soil-less.

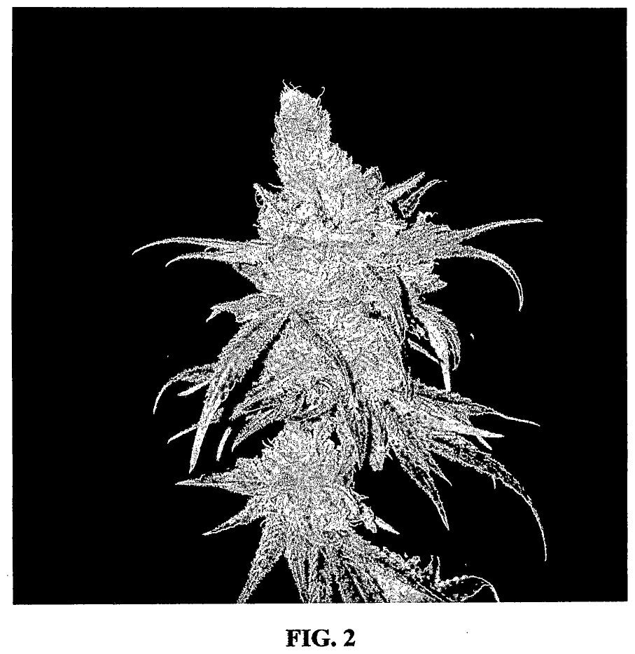

FIG. 3 is a photograph of the new variety ‘DRG2’ at 67 days indoor live soil.

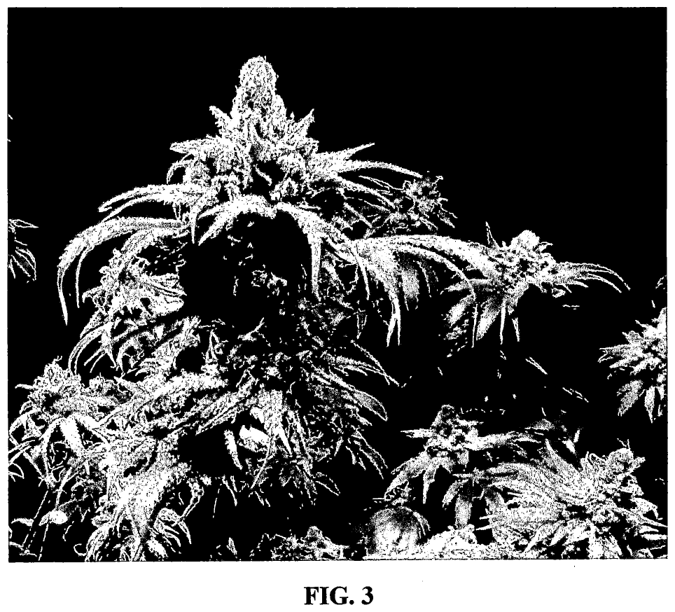

FIG. 4 is a photograph of the new variety ‘DRG2’ at 28 days outdoor full sun vegetative.

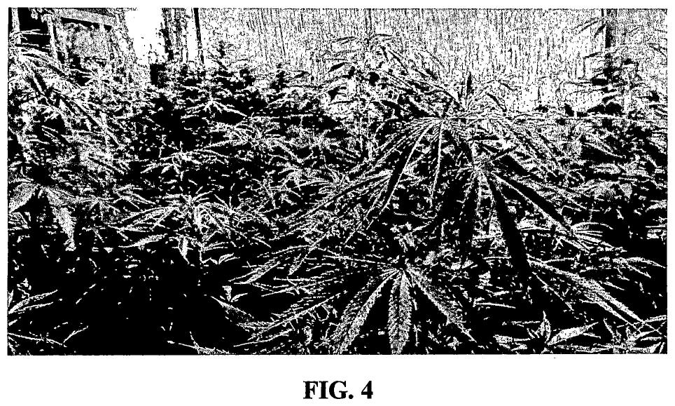

### DETAILED BOTANICAL DESCRIPTION

The following detailed description sets forth the distinctive characteristics of ‘DRG2’. The data which defines these characteristics was collected from asexual reproductions of the original selection. Dimensions, sizes, colors, and other characteristics are approximations and averages set forth as accurately as possible. The plant history was taken on mother plants approximately 1 to 3 years of age, and the descriptions relate to plants grown in Mendocino, CA and Humboldt County, CA.
Type: Herbaceous tap-rooted annual.
Classification:
   a. Family.—Cannabaceae.
   b. Genus.—Cannabis.
   c. Species.—sativa.
   d. Common Name.—Marijuana.
Plant:
   General.—.
   a. Origin.—Mendocino Farms, CA.
   b. Parentage.—. i. Male parent: ‘Old Dave’. ii. Female parent: ‘Blue Dream’.
   c. Growth habit.—Vigorous and open.
   d. Average height.—60″-72″.
   e. Average spread.—48″-60″.
   f. Growth rate.—Fast with long flowers.
   g. Branching characteristics.—Moderate, long, and upswept.
   h. Length of primary lateral branches.—40″-60″ (100-150 cm).
   i. Quantity of primary lateral branches.—>18.
   j. Color.—Green/beige with maroon veining.
   k. Texture.—Ribbed and rough.
   l. Strength.—Moderate.
   m. Internode length.—2.5″-5″ (5-13 cm).
   n. Cold hardiness.—Semi-hardy.
Foliage:
   General.—An open, blue-green “Christmas Tree” canopy with a Sativa-leaning appearance.
   a. Leaf.—Long, open fingers.
   b. Arrangement.—Opposite decussate to alternate in flowering.
   c. Leaf color (Top side).—Blue green.
   d. Leaf color (Under side).—Light green.
   e. Leaf arrangement.—Opposite decussate to alternate in flowering.
   f. Leaf shape.—5 finger sessile leaflets.
   g. Leaf margins.—Serrate.
   h. Undulation of margin.—Typical to species, varies by leaf size.
   i. Leaf apices.—Pointed attenuata.
   j. Leaf bases.—Sagittate.
   k. Leaf width.—8-18 cm.
   l. Leaf length.—11-22 cm.
   m. Texture of top and bottom surfaces.—Smooth veined and matte fuzzy.
   n. Appearance of top and bottom surfaces.—Matte smooth and matte veined.
   o. Venation type.—Palmate.
   p. Petiole length.—3.5-5 cm.
   q. Petiole color.—Blue/green/maroon.
   r. Petiole texture.—Smooth, bi-color top to bottom.
Inflorescence:
   General.—A Complex Pinnacle.
   a. Natural flowering season.—Mid to late October.
   b. Inflorescence and flower type and habit.—A main branch pinnacle.
   c. Fragrance.—Skunk and fuel with a hint of fruit.
   d. Female flower and bud.—i. Bud shape — Urn panicle. ii. Bud length — 5 cm. iii. Bud diameter — 3 cm. iv. Bract diameter — 0.25 cm. v. Bract length — 0.5-0.8 cm. vi. Bract shape — Ovate. vii. Bract base — Perigonal. viii. Bract texture — Hairy. ix. Stigma length — 0.5-0.6 cm. x. Stigma color — White/cream/gold. xi. Stigma number — 2. xii. Stigma shape — Round hirsute. xiii. Ovary position — Bracts base. xiv. Ovary shape — Ovate. xv. Ovary length — 1/32″+/−(0.1 cm+/−).
Seeds:
   General.—
   a. Shape.—Obtuse.
   b. Length.—0.4 cm+/−.
   c. Width.—0.2 cm+/−.

‘DRG2’ can have a cannabinoid profile as set forth in Table 1 and a terpene profile as set forth in Table 2. Due to the natural variability of chemotypic expression that is commonly observed in Cannabis plants, arising from numerous causes such as cultivation conditions, the values set forth in the tables do not reflect the only possible range of outcomes that can be obtained from plants of the new variety. Thus, these values are merely exemplary of observed values (middle column) and predicted normal variations from the observed values.

TABLE 1
Exemplary Profiles of Key Cannabinoids.

| | Percent | Percent | Percent |
|---|---|---|---|
| THCa | 17.1048 | 21.381 | 25.6572 |
| Δ9THC | 0.928 | 1.16 | 1.392 |
| CBGa | 0.392 | 0.49 | 0.588 |
| CBCa | 0.224 | 0.28 | 0.336 |
| CBG | 0.088 | 0.11 | 0.132 |
| THCVa | 0.0776 | 0.097 | 0.1164 |
| CBDa | 0.0704 | 0.088 | 0.1056 |
| Total THC* | 15.9288 | 19.911 | 23.8932 |
| Total CBD** | 0.0616 | 0.077 | 0.0924 |
| Total Cannabinoids*** | 16.6904 | 20.863 | 25.0356 |

*Total THC = Δ9THC + (THCa * 0.877)
**Total CBD = CBD + (CBDa * 0.877)
***Total Cannabinoids = Total THC + Total CBD +Total CBG + Total THCV + Total CBC +Total CBDV + Δ8THC + CBL + CBN

TABLE 2
Exemplary Profiles of Key Terpenes.

| | Percent | Percent | Percent |
|---|---|---|---|
| Myrcene | 0.568 | 0.71 | 0.852 |
| alpha Pinene | 0.3176 | 0.397 | 0.4764 |
| beta Caryophyllene | 0.2216 | 0.277 | 0.3324 |
| beta Pinene | 0.128 | 0.16 | 0.192 |
| Limonene | 0.0808 | 0.101 | 0.1212 |
| alpha Humulene | 0.076 | 0.095 | 0.114 |
| Linalool | 0.04 | 0.05 | 0.06 |
| alpha Bisabolol | 0.032 | 0.04 | 0.048 |
| Terpineol | 0.016 | 0.02 | 0.024 |
| Nerolidol | 0.0104 | 0.013 | 0.0156 |
| Nerol | 0.008 | 0.01 | 0.012 |
| Total Terpenoids | 1.4984 | 1.873 | 2.2476 |

1. A new and distinct variety of Cannabis plant named ‘DRG2’, as illustrated and described herein.
